(19) United States
(12) Patent Application Publication
(10) Pub. No.: US 2019/0098395 A1
(43) Pub. Date: Mar. 28, 2019

(54) EARPHONE SYSTEM

(71) Applicant: KEEWAVE CORPORATION, Calgary (CA)

(72) Inventor: Adam Keeling, Calgary (CA)

(21) Appl. No.: 15/755,665

(22) PCT Filed: Aug. 23, 2016
(86) PCT No.: PCT/CA2016/050990

§ 371 Date: Feb. 27, 2018

Related U.S. Application Data
(60) Provisional application No. 62/212,812, filed on Sep. 1, 2015.

(51) Int. Cl. H04R 1/10; H02J 7/02; G10K 11/178; H04R 25/00; A61B 5/12; A61B 5/00; A61F 11/14

(52) U.S. Cl. CPC H04R 1/1091; A61F 2011/145; G10K 11/17827; G10K 11/17823; H04R 1/1016; H04R 1/1041; H04R 1/1025; H04R 25/554; H04R 25/558; A61B 5/12; A61B 5/6803; A61F 11/14; G10K 2210/1081; H04R 2420/07; H04R 2460/01; H04R 2225/55; H04R 2225/41; H02J 7/025

(57) ABSTRACT

An earphone system for use by a wearer includes earphone, a microphone, actuation switches, a battery with wireless charging and sensors for sensing biometrics of the wearer in the ear and environmental conditions. The individual earphones are arranged for communication with a portable communications device (PCD) for signal processing and external communication. The system is programed to transmit sounds from the PCD to the wearer, to provide enhanced hearing of sounds detected by the microphone to be transmitted to the wearer in dependence on a hearing test, for noise cancellation, to communicate different sounds to each earphone and for processing of biometrics data obtained by said sensor.

### EARPHONE SYSTEM

This invention relates an earphone system for use by a wearer for transmitting sounds, for providing enhanced hearing, for data collection and processing and for biometrics. The earphone system includes a communications protocol for communication with a smart phone and other devices for signal processing and external communication.

### SUMMARY OF THE INVENTION

According to the present invention there is provided an earphone system for use by a wearer comprising:

a pair of earphones for insertion into the ear canal of the wearer;

a microphone for detecting peripheral sounds around the wearer;

actuation switches on the earphones for actuating control functions;

the earphones having a communication system for communication with a smart phone or similar devices for signal processing and external communication;

at least one biometrics sensor mounted on at least one of the earphones for sensing biometrics of the wearer in the ear;

the communication system being arranged to operate with a downloadable program on the smart phone for:
transmitting sounds from the smart phone to the wearer;
for providing enhanced hearing of sounds detected by the microphone to be transmitted to the wearer;
for noise cancellation;
and for processing of biometrics data obtained by said sensor.

Preferably the earphone is shaped to be inserted into the ear canal in a similar manner to a hearing aid.

Preferably the control switches are arranged such that when a user wishes to hear ambient sound the device audio can be muted and the ambient sound can be captured by the microphone and relayed into the user's ear in a “talk through” mode.

Preferably the programming include thresholds for the noise cancelling, so that an ambient sound which deviates from the average ambient sound is allowed to pass through without cancellation.

Preferably the earphone device is controlled and calibrated by the remote external devices such as a portable communications device.

Preferably aspects of the audio such as the equalization can be individualized to the user's preference.

Preferably the control switches are arranged so as to switch between the two listening modes so that the wearer can take a call with one ear while still able to hear the surroundings with the other ear.

Preferably the control switches are arranged to provide the option of muting the sounds to both ears or just muting one ear well still listening to the sounds in the other.

Preferably the programming is arranged to run a hearing test.

Preferably, before the hearing test begins, the software accesses the devices microphone to measure the ambient noise to insure the individual is in an appropriate quiet environment

preferably the programming is calibrated so that the Enhanced Hearing mode acts like a hearing aid for sounds detected by the microphone.

Preferably all music or streaming audio provides the perfect pitch for the wearer as determined by the hearing test.

Preferably the biometric sensors read the information and send it down to the portable communications device's software app for processing and displaying.

Preferably there is provided a location beacon that is tied to the software app to help locate the device if lost.

Preferably wherein the earphones include wireless charging of batteries where the wireless charging unit also acts as their carry case.

References to the term SmartPhone is intended to include any communications device including many other signal receivers such as Apple (trademark) watch, Goggle Glasses, etc.

The reference used in the specification refer to biometric data. It is intended that this term is not limiting and includes any biological and/or physiological data. That is the intention is to include and data measurable on the body which the medical profession considers to be desirable.

Core indicators can include:

physical activity, steps, stair climbing, etc where level of activity can be measured)

body temp, blood pressure, pulse, heart rhythm, brain waves, blood oxygen levels, etc

the system can be used to send voice or other alerts to patients to take remedial actions based on combination of treatment they are on and values that the sensors can monitor.

In regard to the sensing system, voice-patterns can be used in the sensor system for example for authentication. For example, in a military application where someone's implant is removed by the wrong party and inserted into another person so they can tap into the communications stream, then the authentication would be very important. Voice-pattern recognition can be used and linked to other historical biological readings as well as those at time of communication for example Heart rate, blood pressure, etc are not consistent with correct person when matched to voice.

Biological metrics include:

heart rate (to the point of detecting an arrhythmia)

blood pressure

blood oxygen levels

body temperature

interface with smart device including Electronic Medical Record to get physical activity/inactivity measures, steps, stair climbing, sitting too long,

balance/inner-ear problems

Environmental metrics which could be used by first-responders/EMS persons can include:)

external temperature?

atmospheric oxygen?

a measure of someone being in under water for more than a set time period can be used to send an alarm?

Practical alerts can include:

sound or cue for specific appointments (e.g., medical appointments)

sound or cue to take a medication

sound or cue regarding diet/meals for example for diabetics.

The system thus provides an in-ear earphone with three modes of operation:

Headphone—this plays music or audio from another device, such as a phone

Enhanced hearing—ambient sound can be amplified or equalized including hearing aid capabilities on a medical basis or calibrated to correct for the user's hearing loss profile.

Quiet—ambient sound can be suppressed (Noise Cancellation)

The device has a simple touch interface with controls for switching modes, activating voice services such as Siri and controlling income/outgoing streaming communications such as phone calls.

The device is shaped to be inserted into the ear canal in a similar manner to a hearing aid. It may, therefore, be less convenient to quickly remove and replace. However, when a user wishes to hear ambient sound, for example, because someone has started talking to them, the device audio can be muted and the ambient sound can be captured by a microphone and relayed into the user's ear in a “talk through” mode.

There are preferably two microphones, both are on the device. The first is placed somewhere on the exterior to receive sound for the outside environment for the enhanced hearing mode (hearing aid) and noise cancellation. The second is internal directed to the ear canal. It receives sound from within the ear canal, this acts as a feedback for the noise cancellation.

The earphone includes biometric sensors for recording physiological parameters from the wearer. These sensors include:

Accelerometer (Step counter)

Heart rate

Blood Oxygen

Body temperature

Sweat

Electrolyte levels

Hydration level

Lactic acid level

Other metabolites

Bio-impedance

Respiration

Galvanic skin response (GSR)

Optional features include thresholds for the noise cancelling, so that an ambient sound which deviates from the average ambient sound is allowed to pass through without cancellation. For example, a sudden loud siren or alarm deactivates noise cancelling.

The earphone device can be controlled and calibrated by a remote external devices such as a smartphone. Aspects of the audio such as the equalization can be individualized to the user's preference.

### BRIEF DESCRIPTION OF THE DRAWINGS

FIG. 1 is a schematic illustration of the components of the system according to the invention.

FIG. 2 is a graphic showing the programming of the system of figure 1.

### DETAILED DESCRIPTION

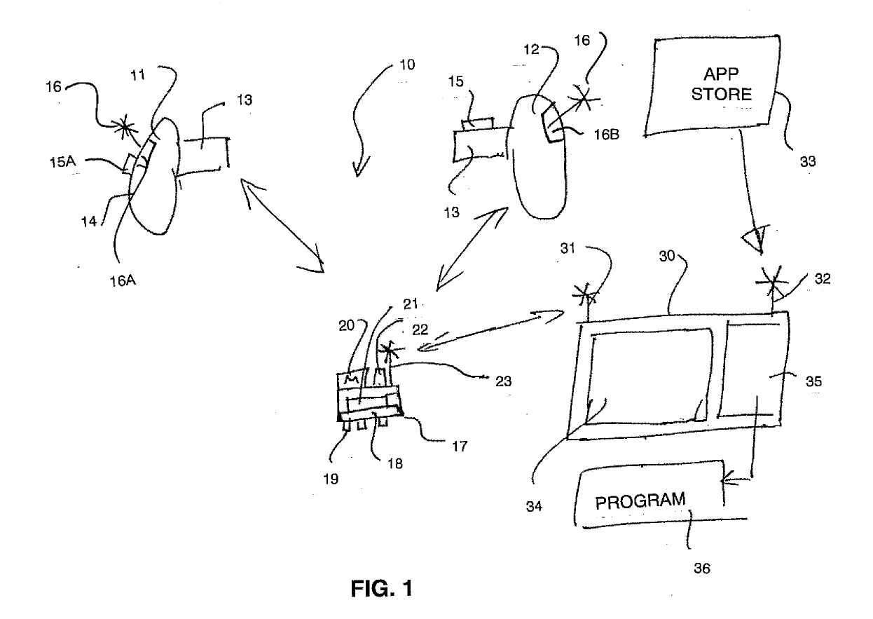

In FIG. 1 is shown a headset generally indicated at 10 with headphones 11 and 12 each having a component 13 for insertion into the ear channel and a component 14 moulded to best fit the ear cavity of the wearer. On one or both of the components 13 is provided a sensor 15 arranged to detect various biometrics all of the wearer and a sensor 15A to detect various environmental conditions, as described in more detail hereinafter.

Each earphones includes processor 16A, 16B which connected by a wireless communication antennas 16 which connects to a centre control unit 17. The control unit includes a central processor 18 operable by manual switches 19. These switches can be provided either on a central unit or on the individual earphones. A microphone the 20 is carried on the central unit 17. A battery 21 is chargeable by an input wireless charging device 22 which receives a power from an associated wireless charging system for example mounted in a carrying case of the headphones. An antenna 23 is provided for wireless communication with a remote portable communications unit (PCU) 30 using conventional communications protocol. The earphones are completely independently wireless each with its own central control unit 17.

The PCU 30 includes an antenna 31, a second antenna 32 for remote communications for example with App Store 33, and input/display screen 34 and a processing unit 35 communication with a program 36 which can be downloaded from the App Store 33.

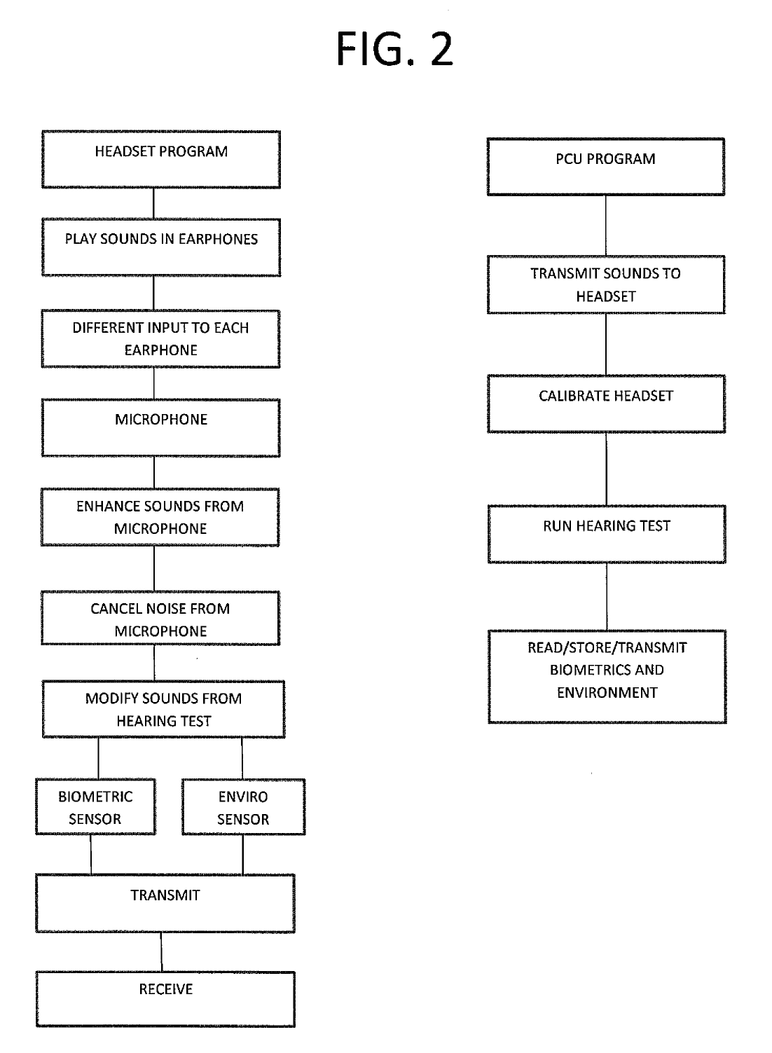

FIG. 2 shows the various aspects of the programs of the headset and the PCU.

The present invention merges three separate industries into one new product. We are merging the hearing aid industry with the headphone industry and the wearables industry. The device fits into the very new Hearables Industry.

The wireless, Bluetooth headphones calibrate to the particular hearing characteristics of the user to give the perfect sound. The headphones also measure biometrics and environmental conditions to let the user know how the body is reacting to the user's workout.

The premise of the arrangement described herein is technical convergence, merging technologies of these separate industries into one product and combined with a software app for the smartphone.

When the device is on, it operates in three modes. Headphone, Enhanced Hearing (Hearing Aid), Quiet.

Headphone Mode: Allows audio input from external device. Playing music, talking on the phone, etc.

Enhanced Hearing Mode: When not playing music, talking on the phone, etc. the device acts as a hearing aid.

Quiet Mode: Stopping all audio inputs and turning on active noise cancellation.

Single push-button controller on each device.

Right ear button (single click): Controls income/outgoing streaming communications (phone calls or other communications). Similar to apple (trademark) headphone, a single-click plays and pauses, takes calls, ends calls.

Right ear button (Held down): Actives voice communication such as Siris

Left ear button (single click): Controls just the left ear, to switch between the two listening Modes. This way the user can take a call with the right ear, well still able to hear the surroundings with the left ear. Or another example would be if the user is listing to music in both ears and some approached the user wishing to talk, the user has the option of muting the music with both ears using the right ear button or just muting the left ear well still listening to music in the right.

Left ear button (Held down): Switches both ear to Quiet Mode. For calibrating the device to one's personal hearing characteristics, there is provided a feature in the software app that runs a hearing test, the same test from a audiologist. The results of the test (audiogram) are uploaded to the device via Bluetooth to calibrate the sound processor. The device now calibrated the Enhanced Hearing mode acts exactly like a hearing aid, and all music or streaming audio provides the perfect pitch.

The device can be FDA certified as a class 2 medical device to be prescribed as a hearing aid for those with mild to moderate hearing loss.

Along with headphone and hearing aid technology combined into the device is the wearable technology. The broad term “biometrics” can be used for this product. The list of biometrics are the following: Accelerometer (Step counter)

Heart rate

Blood Oxygen

Body temperature

Sweat
Electrolyte levels
Hydration level
Lactic acid level
Other metabolites

Bio-impedance
Respiration
Galvanic skin response (GSR) Altimeter

All of which read the information and send it down to the smartphone software app for processing and displaying. The location of all of the sensors in the broadest sense are on the device in the ear.

Other features of the device are that it are waterproof with a minimum ingress protection rating of IP-66.

The audio is THX certified.

The device comes with location beacon that is tied to the software app to help locate the device if lost.

The devices incorporates wireless charging of batteries, rather than having to replace the batteries when charged. The wireless charging unit also acts as their carry case.

This charging case has a battery pack that can charge the device 3-4 times before having to be recharged itself.

The noise cancellation or “Quiet Mode” this uses active noise cancellation. This acts similarly to an earplug to cancel out the noise.

The Bluetooth communication is preferably version 4.2 along with an aptX codec to handle the high bandwidth required although of course other wireless protocol can be used.

### Software App

The system includes a software application for the iPhone (trademark), Apple (trademark) Watch, iPod (trademark) and Android (trademark) smartphones.

The software can be broken down into 8 sections and one small miscellaneous items.

Hearing test

Live Acoustic Calibration

Locator

Biometric readouts

Exercises tracker

Motivation

Initial Setup

Fitness Tests

Other/Misc

### Hearing Test

As described above this feature acts to test one's hearing similar to an audiologist, and upload the results to the devices sound processor. Before the hearing test begins, the software accesses the devices microphone to measure the ambient noise to insure the individual is in an appropriate quiet environment

### Live Acoustic Calibration (For Enhanced Hearing Mode)

This feature allows the user to do live calibration of the sound during Enhanced hearing mode. A good example is the work done by Doppler Labs.

The device has the ability to adjust the Bass, Midrange, and Treble and also apply acoustic effects to the enhanced hearing sound

### Locator

As explained above, this feature allows the user to find their device if lost, similar to the Tile product

### Biometric Readouts

The readouts are a full page window on the app that displays the active biometrics such as:

Heart rate

Resting heart rate

VO2

Max Body temperature

BMR

Calorie Burn Rate

Exercises tracker

the App allows the user to setup their workout and track and display their progress and biometrics once started. The items of the feature are:

Select workout:

Running

Cycling

Hiking

Walking

Treadmill

Ability to set the workout intensity (HR Zone) (Jabra app)

Create workout programs

Set training zones (HR zones) (Jabra app)

Time based or;

Rep based intervals

Set goals: makes use of the trainer to increase/decrease intensity

Average Pace

Average Speed

Calorie burn rate

Choose trainer intensity

Create and save training sessions

### Display:

The display on the smart phone using the app can be customized as to what the user wishes to display.

Current heart rate

Max heart rate

Average heart rate

Body temperature

Workout time

What the next training zones coming up is and what the previous zone

was

Time spent in HR zones

Map tracker (RunKeeper app)

Current pace

Average pace

Split pace

Current speed

Average speed

Split speed

Cadence

Timer, stopwatch

Calories burned

Distance

Elevation

Music controls

Workout controls (Pause⇒Resume or Complete)

### Audio Cues and Trainer: (Jabra & RunKeeper App)

User can setup what is readout;
How often the Cues happen: Time or distance or on demand Set volume

reads out any of the above display items, including the following:
Time (current time of day)

Choose default trainer intensity
Volume

Ability to adjust view
Which state to have as the large focus:
   Heart rate
   Intensity Zone
   Workout time
   Interval time
   Workout summaries/achievements

### Motivation

This feature helps the individual stay motivated to stay on track with their training, some of the features are:

Badges
Achieving # steps, km run, etc.

Reminders
Setup in the initial setup process app examples

Trends
View graphical representation any of the biostat trends (Apple Health) (trademark)

Trainer
Linked to goals

### Initial Setup

This feature is the initial setup of the device and software, having the user enter their starting stats among other items.

Setup instructions (Jabra pulse app)
How to images and maybe a short repeating animation
How to turn the device on/off
How to properly put the device in the ear
How to change the size of tips and wings for the proper fit
Connect and audio test device is operating
How to clean it
Link third party apps and upload stats (autosense which apps the user has)
Apple's “Health” (trademark)
Withings (trademark) (upload weight, body fat, blood pressure stats)
Up (upload sleep stats)
Fitday (trademark)
My fitness pal (trademark)

Metrics
Mile or Kilometer
Celsius or Fahrenheit

Body stats
Sex
Height (lbs or kg)
Weight (Can be retrieved from third party apps) (ft/in or cm)
Age

Automatically calculate requirements and baseline stats
Steps required
Sleep required
Healthy Body Fat %
BMR (Base Metabolic Rate)

Set up Goals, while presenting they their current body stats/requirements

Present:
Minimum Steps required
Optimal Body Fat %:
   Overweight Body Fat %
   Healthy Body Fat % (Optimal)
   Athletic Body Fat %
   Unhealthy Body Fat % (the lowest)
Optimal LBM (Lean Body Mass) (lbs kg)
   Low LBM
   Healthy LBM (Optimal)
   Athletic LBM
Sleep required per night
Current LBM (Lean Body Mass)
Current Fat Mass

Goals:
Steps
Body Goals:
   Muscle gain
      Muscle Mass LBM (lbs kg)
   Maintain weight
   Loss Body Fat
      Body Fat %
Auto calculate Calorie Intake (Range with a delta of 300 Cal)

Set default trainer voice
Male
Female
Link to inapp purchase of additional trainers

Setup reminders & Notifications
Training reminders

### Fitness Tests

This feature allows the users to test their physical abilities to standard test and/or to measure certain body stats such as VO2 max. The list of test is as follows:

VO2 Max (Rockport test)
Orthostaticheart rate test
Heart rate recovery
Resting heart rate
Cooper Test
Shuttle Test Challenge
Police Fitness Test Challenges
Military Fitness Test Challenges

### Other

Input food (calories) automatically jumps to a linked app

Automatically start music when exercise starts
Setup link to iTunes music

Automatically ask link to app in setup

Compatible with third party apps
RunKeeper (trademark) app (Send information on HR and Calorie burn rate)
Apple Health (trademark) app (Send information on all bio stats)
Fitday app (trademark) (Send information on calories burned)
Withings (trademark) (Retrieve body information, such as weight and fat %)
And a few of the other most popular health apps used.
   Dev's session is ice Operation:

When the device is On, it operates in three modes. Headphone, Enhanced Hearing (Hearing Aid), Quite.
“Headphone” Mode: Allows audio input from external device. Playing music, talking on the phone, etc.
“Enhanced Hearing” Mode: When not playing music, talking on the phone, etc. the device's as a hearing aid.
“Quiet” Mode: Stopping all audio inputs and turning on active noise cancellation.

Single push-button controller on each device.
Right ear button (single click): Similar to apple (trademark) headphones, single-click plays and pauses, takes calls, ends calls.
Right ear button (Held down): Actives Siris(trademark)
Left ear button (single click): Controls just the left ear, to switch between the two listening Modes. This way the user can take a call with the right ear, well still able to hear the surroundings with the left ear. Or another example is when listing to music in both ears and some approached the user wishing to talk, the user has the option of muting the music with both ears using the right ear button or just muting the left ear well still listening to music in the right.
Left ear button (Held down): Switches both ear to Quiet Mode.

### Device:

### Internal

Device is Medical grade from part to manufacturing

All regulatory compliance for a Class II medical device, for USA and Canada

Bluetooth Smart (v4.2)

Medical quality Microphone and Transducer

Wearable tech (Picked from Wish List)—Need to discuss what Device Labs can accomplish.

THX certification

Rechargeable Lithium Ion battery and comes with a Carry case that wirelessly charges the Devices
Carry case has the capacity to full charge the devices 4-5×
Carry case can be charged by USB

Ingress protection (IP) rating. A minimum IP-67 rating

Impedance (touch sensitive) button

Full suite of wearable tech, which communicates with our software app
Accelerometer (Step counter)
Heart rate
Blood Oxygen
Body temperature
Sweat
   Electrolyte levels
   Hydration level
   Lactic acid level
   Other metabolites
Bio-impedance
   Respiration
   Galvanic skin response (GSR)
Altimeter
Etc. (Results of TeamThink market research)

Locator beacon—Easily find lost devices, showing both a location and a time stamp. A signal detector sends a stronger or weaker signal based on how close the user is to locating the hearing aids.

Isodynamic (aka orthodynamic) transducer

External (Industrial design)
Securing wing, lightly spring loaded. (See examples)
Securing wing is customizable. Can switch out sizes and colours
Nano-coating to repel water
Padding Medical-grade hypoallergenic TPU rubber
Large push-button controller, similar to Bragi Dash (See example)
Indicator light is our Icon
   Light is on: device is in Headphone or enhanced hearing mode
   Light is slowly pulsing: Quiet mode
   Light of: device is off
The speaker tip enters deeper into the ear canal, similar to a hearing aid. “Canalphones” (IEM)
Speaker soft tips are customizable. Can switch between sizes

### Firmware

Frequency tuning (Hearing aid aspect)
Active noise cancellation
Communicate wearable tech to external device
aptX audio codec

As shown in FIG. 2 audio from phone calls, music etc is communicated from the PCD to the earphone system. The sound from the microphone and communicated to the ear phones is processed by the earphone processor in the program. All audio output to the ear phones can be programed in the headset program including the enhancement of the sound, the modification of the sound in dependence on the hearing test and the cancellation of the sound as set forth above. Thus all audio inputs from the earphones microphone 20 is processed internally by the earphones processor 17. No microphone data is sent down to the PCU 30, only biometric and environmental sensor data is sent down to the PCU for processing.

In FIG. 1, there is provided a central processor for the two separate earphones. In an alternative arrangement, the smart phone can provide the functions of the central processor. That is the smart phone is the main user interface. This can control the sound level and communicating information (ie streaming audio) to both earphone devices as well adjusting many other features, such as turning on and adjusting noise cancellation.

It will be appreciated therefore that the processing can occur in one or other of the devices either using the downloaded program in the smart phone as the main processor of providing more processing power and programing in the earphones themselves or in a master one of the earphones with the other acting as a slave device.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. An earphone system for use by a wearer comprising:
a pair of earphones for insertion into the ear canal of the wearer;
a microphone for detecting peripheral sounds around the wearer;
actuation switches on the earphones for actuating control functions; the earphones having a communication system for communication with a portable communications device for signal processing and external communication;
at least one biometrics sensor mounted on at least one of the earphones for sensing biometrics of the wearer in the ear;
the communication system being arranged to cooperate with a downloadable program on the portable communications device for:
transmitting sounds from the portable communications device to the wearer;
for providing enhanced hearing of sounds detected by the microphone to be transmitted to the wearer;
for noise cancellation;
and for processing of biometrics data obtained by said sensor.

2. The system according to claim 1 wherein the earphone is shaped to be inserted into the ear canal in a similar manner to a hearing aid.

3. The system according to claim 1 wherein the control switches are arranged such that when a user wishes to hear ambient sound the device audio can be muted and the ambient sound can be captured by the microphone and relayed into the user's ear in a “talk through” mode.

4. The system according to claim 1 wherein the programming include thresholds for the noise cancelling, so that an ambient sound which deviates from the average ambient sound is allowed to pass through without cancellation.

5. The system according to claim 1 wherein the earphone device is controlled and calibrated by the remote external devices such as a portable communications device.

6. The system according to claim 1 wherein aspects of the audio such as the equalization can be individualized to the user's preference.

7. The system according to claim 1 wherein the control switches are arranged so as to switch between the two listening modes so that the wearer can take a call with one ear while still able to hear the surroundings with the other ear.

8. The system according to claim 1 wherein the control switches are arranged to provide the option of muting the sounds to both ears or just muting one ear while still listening to the sounds in the other.

9. The system according to claim 1 wherein the programming is arranged to run a hearing test.

10. The system according to claim 9 wherein, before the hearing test begins, the software accesses the devices microphone to measure the ambient noise to insure the individual is in an appropriate quiet environment.

11. The system according to claim 9 wherein the programming is calibrated so that the Enhanced Hearing mode acts like a hearing aid for sounds detected by the microphone.

12. The system according to claim 9 wherein all music or streaming audio provides the perfect pitch for the wearer as determined by the hearing test.

13. The system according to claim 1 wherein the biometric sensors read the information and send it down to the portable communications device's software app for processing and displaying.

14. The system according to claim 1wherein there is provided a location beacon that is tied to the software app to help locate the device if lost.

15. The system according to claim 1 wherein the earphones include wireless charging of batteries where the wireless charging unit also acts as their carry case.
